(19) United States
(12) Patent Application Publication
(10) Pub. No.: US 2015/0147417 A1
(43) Pub. Date: May 28, 2015

(54) METHOD AND PHARMACEUTICAL COMPOSITION FOR LIVER FIBROSIS PREVENTION AND/OR TREATMENT

(71) Applicants: Ching-Feng Weng, Hualien (TW); Yi-Chen Chia, PingTung (TW); Chia-Jen Wu, Hualien (TW); Hsiang-i Tsai, Tainan (TW)

(72) Inventors: Ching-Feng Weng, Hualien (TW); Yi-Chen Chia, PingTung (TW); Chia-Jen Wu, Hualien (TW); Hsiang-i Tsai, Tainan (TW)

(73) Assignee: National Dong Hua University, Hualien (TW)

(21) Appl. No.: 14/088,291

(22) Filed: Nov. 22, 2013

(51) Int. Cl. A61K 36/28

(52) U.S. Cl. CPC A61K 36/28
USPC 424/764; 435/375

(57) ABSTRACT

The present invention is related to a method for liver fibrosis prevention and/or treatment and a pharmaceutical composition thereof. The method and the pharmaceutical composition are made by taking the advantage of Blumea lacera ‘efficacy in accumulating lipid in hepatic stellate cells, down-regulating the proliferation of hepatic stellate cells, inhibiting the mobility of hepatic stellate cells, preventing the activation of hepatic stellate cells, and decreasing the synthesis of ECM proteins. The present invention not only provides a novel optional for clinical prevention and treatment of liver fibrosis, but also promotes the industrial value of Blumea lacera.

### BACKGROUND

1. Technical Field

The present disclosure relates to a method and a pharmaceutical composition for liver fibrosis prevention and/or treatment; more specifically, to a method and a pharmaceutical composition for liver fibrosis prevention and/or treatment by using a Blumea lacera extract.

2. Description of Related Art

Liver, an essential organ in human, is composed of parenchymal and non-parenchymal cells (Roberts et al., 2007). Parencymal cells, also called hepatocytes, comprise of 60% in the total cells and 80% volume of liver. Non-parenchymal cells are composed of sinusoidal endothelial cells, kupffer cells, hepatic stellate cells, and hepatic natural kill cells (3-20% each) of the remaining biologically important cells (Malarkey et al., 2005, Santi-Rocca et al., 2009). Liver fibrosis is characterized by excessive deposition of extracellular matrix (ECM) as chronic injury occurred in liver. Many causes, such as chronic alcohol consumption, Hepatitis B and C viruses infection, nonalcoholic steatohepatitis, and genetic disorders, are involved in the progression of fibrosis and even cirrhosis and hepatocellular carcinoma (Farazi and DePinho, 2006). Collagen, secreted by activated myofibroblasts, is the major component of fibrosis. Activated hepatic stellate cells (HSC) is identified as the major collagen-producing cell type in liver (Friedman et al., 1985). Other cell types, such as peribiliary fibroblasts and bone marrow-derived myofibroblasts, are also contributed to fibrosis (Kinnman et al., 2003, Forbes et al., 2004).

Nature products have been applied for medical use for at least 5000 years (Goldman, 2001). In fact, there are more than 85,000 plant species have been applied for medicinal application globally (Balunas and Kinghorn, 2005). Examples of important drugs that obtained from plants are digoxin from foxglove, morphine from poppies, and asprin from salicylic acid in willow bark. There are eighty species in genus of Blumea (B.) that mainly distributed in tropical and subtropical Asia, Africa, and Oceania. The hot water extracts of B. lacera exhibited anti-leukemic activity and suppressed the replication of herpes simplex virus (HSV) types 1 and 2 (Chiang et al., 2004). The flavonoid from the ethanol extracts of B. lacera exhibited anti-bacterial activity (Ragasa et al., 2007). Moreover, the methanol extracts of B. lacera exhibited cytotoxic effects on cancer cells (Uddin et al., 2011). The detailed constituents, including two flavonoids, three monoterpenes, and one triterpenen, have been determined from B. lacera (Chen et al., 2009).

### SUMMARY

One of the objects of the present invention is to provide a method for treating and preventing liver fibrosis and therefore to be helpful in preventing cirrhosis and hepatocellular carcinoma.

Another object of the present invention is to promote the industrial value of B. lacera by evaluating its pharmaceutical application.

In order to achieve the above-mentioned objects, the present invention provides a method for liver fibrosis prevention and/or treatment, comprising: administrating an effective amount of a Blumea lacera extract to an object.

Preferably, said effective amount is 1 to 5 g/60 kg body weight/daily based on the experiments; more preferably, is 1.5 to 2.0 g/60 kg body weight/daily based on the consumable concern daily intake.

Preferably, said effective amount is 100 to 500 μg/ml under in vitro cell culture experiment; wherein said effective amount is based on the volume of culture medium used in each culture of said in vitro cell culture experiment.

Preferably, said liver fibrosis prevention and/or treatment comprising accumulating lipid in hepatic stellate cells, down-regulating the proliferation of hepatic stellate cells, inhibiting the mobility of hepatic stellate cells, preventing the activation of hepatic stellate cells, decreasing the synthesis of ECM proteins, or a combination thereof.

Preferably, the route of said administrating is via oral administration, intravenous injection, or a combination thereof.

Preferably, said Blumea lacera extract is an alcohol extract of Blumea lacera.

Preferably, said alcohol extract is an ethanol extract.

Preferably, said Blumea lacera extract is made by an extraction process; wherein said extraction process comprises the following steps: obtaining a Blumea lacera plant; soaking said Blumea lacera with an ethanol to obtain a mixture; concentrating said mixture to form said extract.

Preferably, said Blumea lacera plant is a whole plant of said Blumea lacera.

Preferably, said soaking is achieved by putting said Blumea lacera into a bath of said ethanol.

Preferably, said ethanol is of a concentration of 90 to 95% (v/v).

Preferably, said soaking is performed for 120 to 168 hours.

Preferably, said soaking is performed at 26 to 28° C.

Preferably, said mixture is filtered before being concentrated.

Preferably, said concentrating is achieved by reduced pressure.

Preferably, said extraction process further comprises the step of dissolving said extract in dimethyl sulfoxide (DMSO), absolute ethanol, anhydrous ethanol, or a combination thereof after concentration.

The present invention also provides a pharmaceutical composition for liver fibrosis prevention and/or treatment, comprising an effective amount of a Blumea lacera ethanol extract and a pharmaceutically acceptable carrier; wherein said effective amount is 1 to 5 g/60 kg body weight/day.

Preferably, said liver fibrosis prevention and/or treatment comprising accumulating lipid in hepatic stellate cells, down-regulating the proliferation of hepatic stellate cells, inhibiting the mobility of hepatic stellate cells, preventing the activation of hepatic stellate cells, decreasing the synthesis of ECM proteins, or a combination thereof.

Preferably, the administration route of said pharmaceutical composition is via oral administration, intravenous injection, or a combination thereof.

Preferably, said Blumea lacera ethanol extract is made by an extraction process; wherein said extraction process comprises the following steps: obtaining a Blumea lacera plant; soaking said Blumea lacera with an ethanol at 26 to 28° C. for 120 to 168 hours to obtain a mixture; concentrating said mixture and affording an extract.

In light of the foregoing, the present invention provides a method for liver fibrosis prevention and/or treatment by using an extract of Blumea lacera and a pharmaceutical composition containing the same. This is another successful instance of applying nature product in medical uses. The disclosure of the present invention also shows a novel medical application of Blumea lacera.

### BRIEF DESCRIPTION OF THE DRAWINGS

FIG. 1 shows the lipid accumulation in NHSC and THSC; NHSC and THSC were treated with 100 (BL100), 250 (BL250), and 500 μg/ml (BL500) of BL extract or curcumin (25 μM) for 12 (A) and 24 h (B). Cells were fixed and stained with Oil red O stock solution, and then counter-stained with Hematoxylin.

FIG. 2 shows the FASN level in BL extract treated NHSC and THSC; NHSC and THSC (5×10⁵ cells) were seeded in 6 cm dish and grown till 80% confluence, and then incubated with 100 (BL100), 250 (BL250), and 500 μg/ml (BL500) of BL extract or curcumin (Cur, 25 μM) for 12 and 24 h. Protein expression level was determined by western blotting and normalized with β-actin. Data are normalized to normal control (N) and expressed as mean±SEM in triplicate determinations. a, vs. N; b, vs. Cur. *, p<0.05; **, p<0.01; ***, p<0.001.

FIG. 3 shows that the viability of NHSC and THSC after BL extract treatment; NHSC and THSC (1×10⁴ cells) were seeded in 96 well plates for overnight and then treated with 100 (BL100), 250 (BL250), and 500 μg/ml (BL500) of BL extract for additional 24 h. The cell viability was analyzed by MTT assay. The results of OD value were measured at 570 nm by ELISA reader. Data are normalized to normal control and represented as mean±SEM in triplicate determinations. **, p<0.01; ***, p<0.001.

FIG. 4 shows the expression levels of p21, Cyclin D1 and Cdk6 in BL extract-treated NHSC and THSC; NHSC and THSC (5×10⁵ cells) were seeded in 6 cm dish and grown till 80% confluence, and then incubated with (BL100), 250 (BL250), and 500 μg/ml (BL500) of BL extract or curcumin (Cur, 25 μM) for 12 and 24 h. Protein expression level was determined by western blotting and normalized with β-actin. Data are normalized to normal control (N) and expressed as mean±SEM in triplicate determinations. a, vs. N; b, vs. Cur. *, p<0.05; **, p<0.01; ***, p<0.001.

FIG. 5 shows the cell mobility of BL extract-treated NHSC and THSC; NHSC and THSC (3.5×10⁴ cells) were seeded in 6 well plates with inserts. Cells were treated with 100 (BL100), 250 (BL250), and 500 μg/ml (BL500) of BL extract for 24 h. The number of cells in the denuded zone was quantitated after 0, 12, and 24 h incubation time by Olympus CKX-41inverted microscope. The pictures of the initial wounded monolayer were compared with the corresponding pictures of cells at the end of incubation.

FIG. 6 shows the expression level of F-actin in BL extract-treated NHSC and THSC. Both NHSC and THSC were treated with 100 (BL100), 250 (BL250), and 500 μg/ml (BL500) of BL extract for 24 h. Cells were fixed and stained for F-actin by rhodamine phalloidin (green color) and nuclei was visualized by Hoechst 33342 (blue color).

FIG. 7 shows the expression levels of TGF-β1 and TGF-β R1 in BL extract-treated NHSC and THSC. NHSC and THSC (5× 105 cells) were seeded in 6 cm dish and grown till 80% confluence, and then incubated with 100 (BL100), 250 (BL250), and 500 μg/ml (BL500) of BL extract or curcumin (Cur, 25 μM) for 12 and 24 h. Protein expression level was determined by western blotting and normalized with β-actin. Data are normalized to normal control (N) and expressed as mean±SEM in triplicate determinations. a, vs. N; b, vs. Cur. *, p<0.05; **, p<0.01; ***, p<0.001.

FIG. 8 shows the expression levels of pSmad 2 and 3, and Smad 4 and 7 in BL extract-treated NHSC and THSC. NHSC and THSC (5×10⁵ cells) were seeded in 6 cm dish and grown till 80% confluence, and then incubated with 100 (BL100), 250 (BL250), and 500 μg/ml (BL500) of BL extract or curcumin (Cur, 25 μM) for 12 and 24 h. Protein expression level was determined by western blotting and normalized with β-actin. Data are normalized to normal control (N) and expressed as mean±SEM in triplicate determinations. a, vs. N; b, vs. Cur. *, p<0.05; **, p<0.01; ***, p<0.001.

FIG. 9 shows the expression levels of Col 1, α-SMA and MMP2 were significantly reduced in BL extract-treated NHSC and THSC. NHSC and THSC (2 or 5×10⁵ cells) were seeded in 6 cm dish and grown till 80% confluence, and then incubated with 100 (BL100), 250 (BL250), and 500 μg/ml (BL500) of BL extract or curcumin (Cur, 25 μM) for 12 and 24 h. Protein expression level was determined by western blotting and normalized with β-actin. Data are normalized to normal control (N) and expressed as mean±SEM in triplicate determinations. a, vs. N; b, vs. Cur. *, p<0.05; **, p<0.01; ***, p<0.001.

FIG. 10 shows the expression level of TIMP2 was significantly reduced in BL extract treated NHSC and THSC. NHSC and THSC (2×10⁵ cells) were seeded in 6 cm dish and grown till 80% confluence, and then incubated with 100 (BL100), 250 (BL250), and 500 μg/ml (BL500) of BL extract or curcumin (Cur, 25 μM) for 24 h. Protein expression level was determined by western blotting and normalized with β-actin. Data are normalized to normal control (N) and expressed as mean±SEM in triplicate determinations. a, vs. N; b, vs. Cur. *, p<0.05; **, p<0.01; ***, p<0.001.

### DETAILED DESCRIPTION

Various Blumea lacera extracts have been showed potential in medical uses such as anti-leukemic activity, suppressing herpes simplex virus (HSV), anti-bacterial activity, cytotoxic effects on cancer cells but, before the present invention, no one has ever known that Blumea lacera has the efficacy in liver fibrosis prevention and/or treatment. The researches of the present invention proved that an extract of Blumea lacera of the present invention showed the abilities of accumulating lipid in hepatic stellate cells, down-regulating the proliferation of hepatic stellate cells, inhibiting the mobility of hepatic stellate cells, preventing the activation of hepatic stellate cells, and decreasing the synthesis of ECM proteins; therefore is pharmaceutically applicable in preventing and/or treating liver fibrosis.

The term of “prevention” or “preventing” is herein referred to avoid an object from the occurrence or onset of a disease, an illness, or an adverse symptom. The term of “treatment” or “treating” is herein referred to eliminate, stop, or reduce the progress of a disease, an illness, or an adverse symptom in an object.

The term of “effective amount” is herein referred to an amount that is sufficient to perform the aforesaid prevention and/or treatment. In terms of in vitro cell culture experiments, the effective amount is defined as “μg/ml” based on the total volume of cell culture medium used in each culture. In terms of animal model experiments, the effective amount is defined as “g/60 kg body weight/day”. In addition, the effective amount collected from in vitro cell culture experiments can be transformed into a reasonable effective amount for animals by the following calculation:
Generally, “μg/ml” (effective amount based on in vitro cell culture experiments)=“mg/kg body weight/day” (effective amount for mouse); and the metabolism rate of mice is 6 times fast compared to human (Reagan-Shaw et al., 2008).
Therefore, if the effective amount based on in vitro cell culture experiments is 500 μg/ml, then the effective amount for mouse shall be 500 mg/kg body weight/day (˜0.5 g/kg body weight/day). And then the effective amount for human shall be 5 g/60 kg body weight/day after conversion followed abovementioned metabolic rate.
According the examples in the following paragraphs, the effective amount based on in vitro cell culture experiments is 100 to 500 μg/ml; therefore, the reasonable effective amount for human shall be 1 to 5 g/60 kg body weight/day; furthermore, by referring to the other related researches (Reagan-Shaw, Nihal and Ahmad, 2008), the effective amount for human is preferably modified as 1.5 to 2 g/60 kg body weight/day.

The Blumea lacera extract of the present invention is made by the following steps: obtaining a Blumea lacera plant; soaking said Blumea lacera with an alcohol to obtain a mixture; concentrating said mixture to form said extract. In a preferable embodiment of the present invention, a whole plant of Blumea lacera is used for extraction. In a preferable embodiment, said alcohol is ethanol. Preferably, an ethanol of a concentration of 90 to 95% (v/v) is used for extraction.

In a preferable embodiment of the present invention, said Blumea lacera plant is dried and cut into pieces before soaking. In an alternative embodiment of the present invention, said soaking is achieved by putting said Blumea lacera plant into a bath of said alcohol. Preferably, said Blumea lacera plant is soaked in said alcohol at 26 to 28° C. for 120 to 168 hours.

In a preferable embodiment of the present invention, said mixture is filtered before being concentrated. Said mixture can be filtered by any manner known in the art, for instance, bag filtration, filter-press filtration, vane filtration, cross-flow filtration, centrifugal filtration, Don filtration, hydraulic cyclone filtration, or ultrafiltration.

In a preferable embodiment of the present invention, said concentrating is achieved by reduced pressure. It is not necessary to limit the pressure for said concentrating as long as it is effective to remove the solvent of the mixture.

In an alternative embodiment of the present invention, said extract is re-dissolved in dimethyl sulfoxide (DMSO), absolute ethanol, abhydrous ethanol, or a combination thereof. Alternatively, other solvents with better biocompatiblity known in the art can also be used for dissolving, storing and/or administrating said extract.

In one aspect of the present invention, a method for liver fibrosis prevention and/or treatment is provided. The method comprises administrating an effective amount of an aforesaid Blumea lacera extract to an object. Said object may have a liver fibrosis or have a potential risk of liver fibrosis. The route of said administrating may be via oral administration, intravenous injection, or a combination thereof. Said effective amount is 100 to 500 μg/ml based on in vitro cell culture experiment, or 1 to 5 g/60 kg body weight/day based on the aforesaid calculation; or more preferably, is 1.5 to 2.0 g/60 kg body weight/day.

In another aspect of the present invention, a pharmaceutical composition for liver fibrosis prevention and/or treatment is provided. The pharmaceutical composition comprises an effective amount of an aforesaid Blumea lacera ethanol extract and a pharmaceutically acceptable carrier; wherein said effective amount is 1 to 5 g/60 kg body weight/day.

In a preferable embodiment, said pharmaceutical composition comprises 1×10⁻² to 5×10⁻² wt % of aforesaid Blumea lacera ethanol extract; and 1×10⁻¹ to 5×10⁻¹ wt % of a pharmaceutically acceptable carrier; wherein said wt % is based on the total weight of said pharmaceutical composition.

Said pharmaceutically acceptable carrier includes but not limited: water, alcohol, glycerol, chitosan, alginate, chondroitin, Vitamin E, mineral oil, dimethyl sulfoxide (DMSO), or a combination thereof.

The following embodiments are recited for further explaining the advantages of the present invention but not for limiting the claim scope of the present invention.

### Example 1

### Preparation of Blumea lacera Extract of the Present Invention

Blumea lacera (BL; Burm. f.) DC. (Compositae) were collected from Ping Tung County, Taiwan in March 2011, and a voucher specimen was deposited in the Department of Food Science & Technology, Tajen University, Pingtung, Taiwan. The air-dried whole plant (15 kg) were cut into small pieces and soaked with ethanol (40 L×3) at room temperature for 144 hours, then filtered by filter-press filtration. The combined filtrates were concentrated and the solvent was removed under reduced pressure to afford the BL crude extract (1077 g). BL crude extract was dissolved in DMSO. In addition, curcumin (Sigma-Aldrich, Saint-Louis, Mo., USA) was also dissolved in absolute ethanol for taking as a control for the following experiments.

### Example 2

### Materials and Methods

### Hepatic Stellate Cells (HSC) Collection and Induction

Non-induced hepatic stellate cells (NHSC, purity >95%) were isolated from male Sprague-Dawley rat liver, and Thioacetamide (TAA; Fluka Chemie GmbH, Buchs, Switzerland)-induced hepatic stellate cells (THSC, purity >95%) were isolated from TAA-induced fibrotic liver of male Sprague-Dawley rat (Chang et al., 2009). Both cells were maintained in complete DMEM containing 10% heat-inactivated FBS and 1% penicillin/streptomycin.

### Oil Red O Staining

NHSC and THSC cells (1×10⁵ cells) were seeded in 6 well plates and incubated at 37° C. overnight. Then, cells were treated with various concentrations of BL extract (100, 250, and 500 μg/ml) or curcumin (25 μM) for 12 and 24 h. Cells were washed twice with PBS and fixed with pre-warmed 3.7% paraformaldehyde for 10 min. Cells were then stained with diluted Oil red O stock solution 0.5% (w/v) in isopropanol (Fluka; buffer/water=3/2) at room temperature for 1 h under the dark. Cells were washed with 50% isopropanol and counter-stained with Hematoxylin (Sigma-Aldrich) for 1 min. Results were photographed using Olympus CKX-41 (Olympus Corporation, Tokyo, Japan).

### MTT Assay

The 3-(4,5-dimethylthiazol-2-yl)-2,5-diphenyltetrazolium bromide (MTT, Invitrogen) is a colorimetric based assay that is performed to analyze the proliferation of cells. NHSC and THSC (1×10⁴ cells) were seeded in 96 well plates for overnight. Cells were treated with various concentrations of BL extract (100, 250, and 500 μg/ml) for additional 24 h, after incubation 20 μl (5 mg/ml) of MTT solution was added per well and further incubated for 4 h. The media was removed, and formazan was solubilized by adding 100 μl/well of DMSO (Sigma-Aldrich) and OD was measured at 570 nm using a microplate reader (ELISA reader, Thermo Labsystems). Percentage of viable cells was estimated by comparing with untreated control cells.

### Wound Healing Assay

NHSC and THSC (3.5×10⁴ cells) were seeded in 6 well plates with inserts. Inserts were removed 24 h after, and cells were washed with PBS and then treated with various concentrations of BL extract (100, 250, and 500 μg/ml) or curcumin (25 μM). Wound closure was monitored and photographed from 0 to 24 h using an Olympus CKX-41 inverted microscope. To quantify the migrated cells, the pictures of the initial wounded monolayer were compared with the corresponding pictures of cells at the end of incubation.

### Immunofluorescence Assay

NHSC and THSC (3×10³ cells) were seeded in black 96 well imaging plates (PerkinElmer) for 16 h. Cells were incubated with various concentrations of BL extract (100, 250, and 500 μg/ml) for additional 24 h. Cells were blocked with 5% BSA/PBS solution at room temperature for 30 min and fixed with 3.7% formaldehyde for 5 min, and then permeabilized with 0.1% Triton-X 100 for 5 min. Finally, cells were stained with Rhodamine Phallodin (Sigma-Aldrich) for 15 min. Before acquiring the images, cells were stained with Hoechst 33342 (Sigma-Aldrich) for 15 min and observed under BD pathway TM instrument (BD Bioscience).

### Western Blotting

NHSC and THSC (5×10⁵ cells) were seeded in 6 cm dish and grown till 80% confluence, and then incubated with various concentrations of BL extract (100, 250, and 500 μg/ml) or curcumin (25 μM) for 12 and 24 h. Cells were collected and lysed with RIPA buffer (150 mM NaCl, 0.1% Triton X-100, 0.5% sodium deoxycholate, 0.1% SDS, 50 mM Tris-HCl, pH 8.0). Protease inhibitor (Roche Applied Science, Mannheim, Germany) containing pancreas extract, thermolysin, chymotrypsin, trypsin, and papain was mixed with 50 ml RIPA buffer before use. Protein samples (30 μg/well) were loaded and separated with SDS-PAGE, and then transferred to PVDF membrane (PerkinElmer, Turku, Finland). The membrane was blocked with 5% non-fat milk in TBST (20 mM Tris-HCl, pH 7.4, 137 mM NaCl, and 0.05% Tween-20) at room temperature for 1 h. The membranes were incubated with primary antibodies (Table 1) at 4° C. overnight. The membranes were washed 3 times with TBST and then incubated with horseradish peroxidase (HRP)-conjugated secondary antibodies at room temperature for 1 h. Following incubation with HRP-conjugated secondary antibodies, membranes were treated with ECL reagents (PerkinElmer) for 1 min, and results were developed on LAS-3000 film (Fujifilm, Tokyo, Japan).

TABLE 1
Primary antibody and secondary antibody

| | MW | Dilution | |
|---|---|---|---|
| Antibody | (kDa) | multiple | Company |
| Cyclin D1 (Monoclonal) | 34 | 1:1000 | Cell Signalling |
| p-AMPK (Monoclonal) | 63 | 1:1000 | Cell Signalling |
| p-Smad 2 (Ser465/467) | 60 | 1:1000 | Cell Signalling |
| p-Smad 3 (Ser423/425) | 52 | 1:1000 | Cell Signalling |
| FASN (monoclonal) | 289 | 1:1000 | Cell Signalling |
| α-SMA (Polyclonal) | 43 | 1:5000 | Millipore |
| Smad 2 (Polyclonal) | 60 | 1:1000 | GeneTex |
| Smad 3 (Polyclonal) | 52 | 1:1000 | GeneTex |
| Smad 4 (Polyclonal) | 60 | 1:1000 | GeneTex |
| Smad 7 (Polyclonal) | 44 | 1:1000 | GeneTex |
| Cdk6 (Polyclonal) | 36 | 1:1000 | GeneTex |
| TGF-β1 Ligand (Polyclonal) | 44 | 1:1000 | GeneTex |
| Col I (Polyclonal) | 129 | 1:1000 | GeneTex |
| TGF-β RI (Polyclonal) | 53 | 1:1000 | Santa Cruz |
| □β-actin (Monoclonal) | 43 | 1:5000 | Sigma |
| Goat anti-Mouse (IgG) | | 1:20000 | GE |
| Goat anti-Rabbit (IgG) | | 1:50000 | GE |

### Gelatin Zymography

The activity of MMP2 was assayed by gelatine zymography, which can detect the active form and latent form of MMP2. NHSC and THSC (2×10⁵ cells) were seeded in 6 well plates and incubated for 70 to 80% confluence. Cells were starved in DMEM containing 0.1% BSA for 6 h, and then treated with various concentrations of BL extract (100, 250, and 500 μg/ml) or curcumin (25 μM) for 12 and 24 h. Media were collected and centrifuged at 12,000×g for 30 min at 4° C. The supernatants were collected and protein concentration was quantified by Bradford dye (Bio-Rad). 8% SDS-PAGE gels were prepared containing 10% gelatin. Proteins were pre-heated at 55° C. with 2× loading dye (0.125 M Tris-HCl, pH 6.8, 4% SDS, 0.04% Bromophenol blue, 20% Glycerol). 7 μg of protein sample was loaded into gel and electrophoresis separation was performed at 80V for 4 h. After electrophoresis, gel was washed 2 times in 50 mL of 2.5% Triton X-100 per gel, and then incubated in developing buffer (0.05 M Tris-HCl, pH 8.8, 5 mM CaCl₂, 0.02% NaN₃) for 16 h at 37° C. Finally, gel was stained in 0.1% Coomassie blue R-250 (Bio-Rad) for 4 h and then destained by fixing buffer (45% methanol, 10% acetic acid). Gels were scanned using Epson scanner and quantified using multi-gauge software (Fujifilm).

### Statistical Analysis

Results are presented as means±SEM. Data were analyzed with one-way ANOVA Turkey test for multiple-group comparisons. The p values less than 0.05 were considered statistically significant.

### Example 3

### Lipid Accumulation were Restored in BL Extract-Treated NHSC and THSC Cells

Loss of lipid droplets in activated hepatic stellate cells (HSC) is an important phenomenon compared to that of quiescent HSC. In this example, the BL extract's effect on restoring lipid droplets accumulation in hepatic stellate cells was tested.

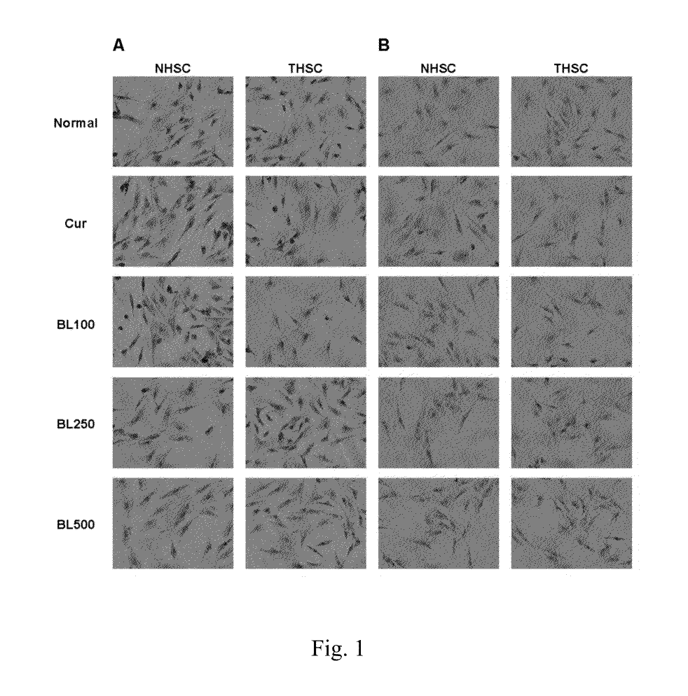

Two kinds of hepatic stellate cells were used; that is, Non-chemical induced hepatic stellate cells (NHSC), and thioacetamide-induced hepatic stellate cells (THSC) for imitating a naturally-occurred activated HSC. Both NHSC and THSC were prepared as set forth in the aforesaid Example 2 and treated with various concentrations of BL extract (100, 250, and 500 μg/ml) or curcumin for 12 and 24 h, and then stained with Oil red O for the detection of lipid droplet (FIG. 1).

The results showed that increased lipid droplets were observed in NHSC and THSC treated with BL extract for 12 and 24 h. More lipid droplets were accumulated in NHSC after BL extract treatment compared to that of THSC. Fewer lipid droplets were increased in cells treated with curcumin compared to that BL extract treated cells.

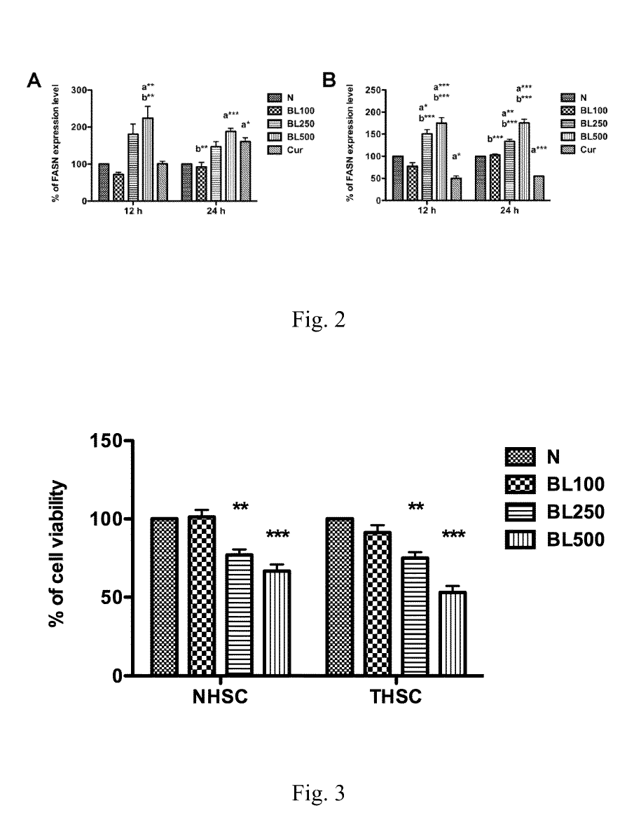

Fatty acid synthase (FASN) is important of lipid accumulation in NHSC and THSC; therefore, the FASN level was also tested in this Example. The results showed that FASN level was significantly increased in BL250 (THSC) and BL500 (both NHSC and THSC) treated cells (FIG. 2). Taken together, more lipid droplets were accumulated in cells treated with BL extract through increase FASN level. Better lipid droplets restored ability of BL extract than that of curcumin.

### Example 4

### Proliferation and Cell Cycle Related Proteins of NHSC and THSC were Significantly Inhibited after BL Extract Treatment

Quiescent hepatic stellate cells (qHSC) are activated and proliferated during chronic liver injury. These activated HSC further migrate to the injured site and regulate subsequent progression of liver fibrosis. Therefore, the BL extract's effect on the proliferation and migration of NHSC and THSC was tested.

Cells were treated with various concentrations of BL extract (100, 250, and 500 μg/ml) for 24 h and proliferation rate was analyzed by MTT assay (FIG. 3). The results showed that proliferation of NHSC and THSC was significantly inhibited in BL250 (both cells, p<0.01) and BL500 (both cells, p<0.001) treated cells.

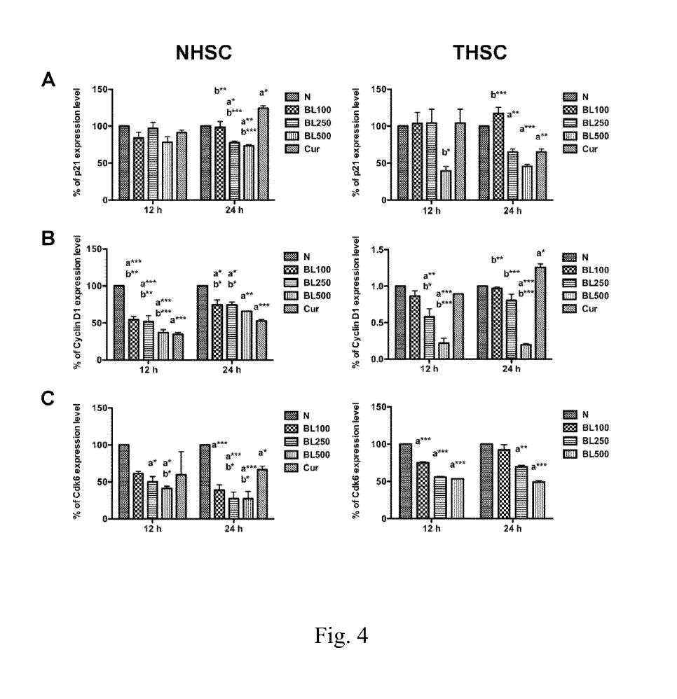

We further analyzed expression level of cell cycle-related proteins, such as p21, cyclin D1, and Cdk6 (FIG. 4). The p21 level was not affected at 12 h in NHSC and THSC after BL extract treatment (FIG. 4A). A significant reduction of p21 at 24 h after BL250 (NHSC, p<0.05; THSC, p<0.01) and BL500 (NHSC, p<0.01; THSC, p<0.001) treatment as compared to that of normal control was observed.

Cyclin D1 and CdK6 are important proteins for cell cycle start in mammalian cells (FIG. 4B). The protein level of cyclin D1 was significantly decreased in NHSC that were treated with BL100, BL250, and BL500 (both 12 and 24 h-treated groups were p<0.05), compared to that of normal control. The cyclin D1 level was also significantly decreased in THSC that treated with BL250 (12 h, p<0.01) and BL500 (both 12 and 24 h, p<0.001). A significant reduction of Cdk6 levels in NHSC and THSC that were treated with BL100 (THSC, p<0.001), BL250 (NHSC, p<0.05; THSC, p<0.001), and BL500 (NHSC, p<0.05; THSC, p<0.001) for 12 h was observed (FIG. 4C). Similar results of reduction were also observed in cells treated with BL100 (NHSC, p<0.001), BL250 (NHSC, p<0.001; THSC, p<0.01), and BL500 (NHSC, p<0.001; THSC, p<0.001) for 24 h. According to these results, BL extract inhibit proliferation through down regulation of cyclin D1 and Cdk6 levels in NHSC and THSC.

### Example 5

### Cell Mobility of NHSC and THSC were Influenced after BL Extract Treatment

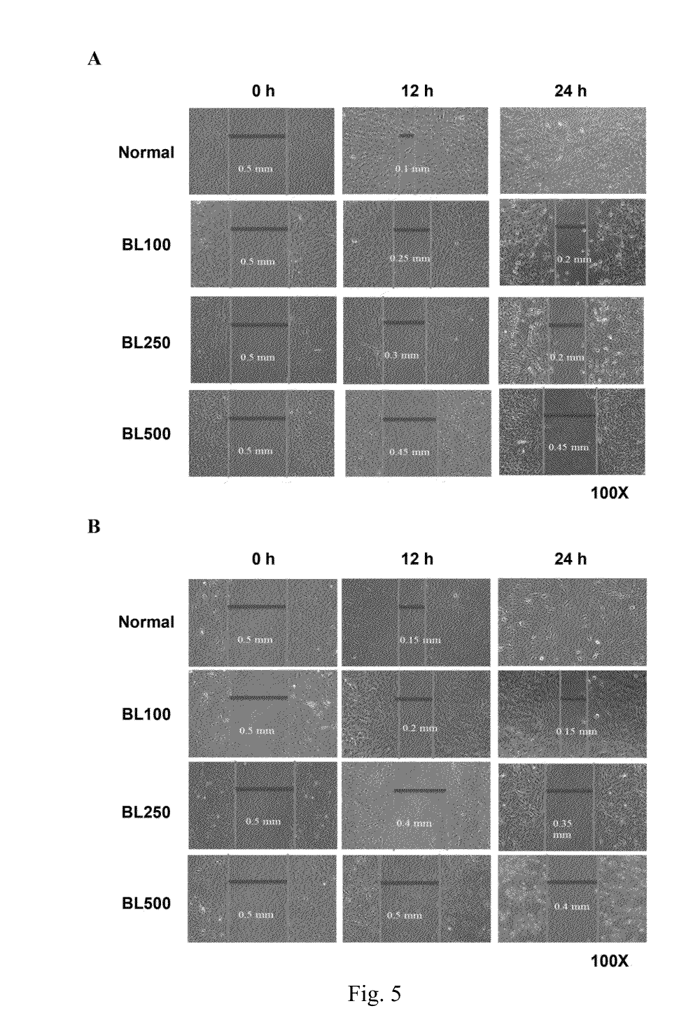

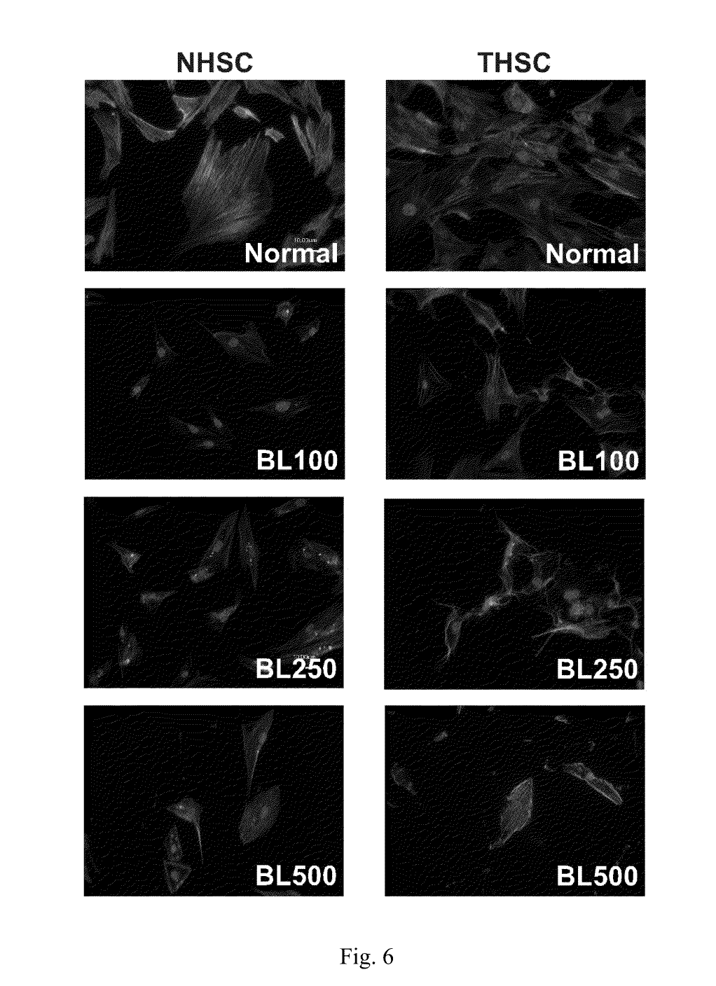

The cell mobility of NHSC and THSC was analysed by wound healing assay (FIG. 5) as recited in the aforesaid Example 2. The blank area was sealed in NHSC and THSC after 24 h incubation. However, a dose dependent manner of inhibition of cell mobility was observed in NHSC and THSC that treated with different concentrations of BL extract.

We also checked the expression pattern of F-actin stress fibers in BL extract-treated cells (FIG. 6). The results showed that NHSC and THSC exhibited well-organized bundles of F-actin stress fibers. After BL extract treatment, the F-actin stress fibers expression level was decreased. Taken together, cell mobility and formation of F-actin stress fibers was influenced in NHSC and THSC that treated with BL extract.

### Example 6

### TGF-β1, TGF-β R1 and Smad Proteins were Significantly Reduced in BL Extract-Treated NHSC and THSC

TGF-β1, TGF-β R1, and Smad proteins are also known to be related to the activation of quiescent HSC. TGF-β1 not only promotes quiescent HSC to active phenotype, but also stimulates the synthesis of ECM proteins. TGF-β1 binds to TGF-β RII and then activates TGF-β R1, which further induced the phosphorylation of Smad2/3. The phosphorylated Smad2/3 then forms a complex with Smad4 and migrates into the nucleus to regulate various genes.

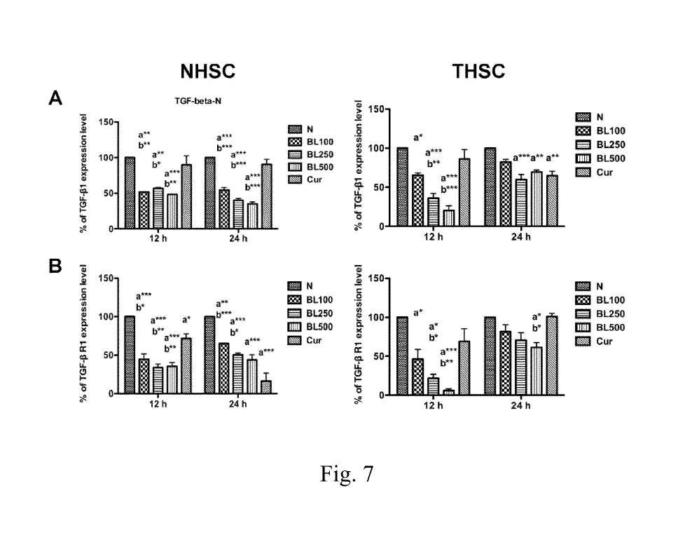

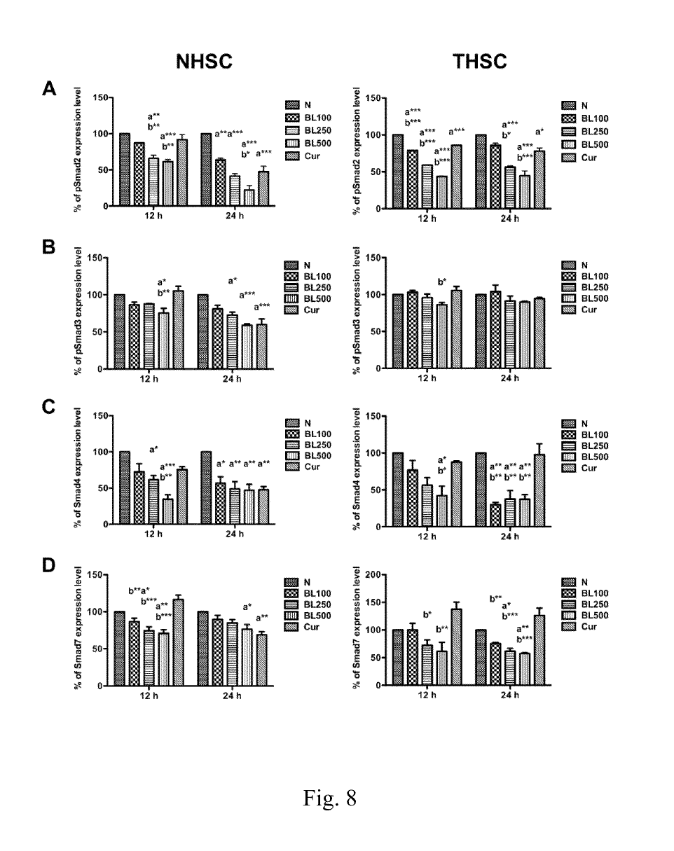

We further analyzed whether TGF-β1 and TGF-β R1 were affected in BL extract-treated NHSC and THSC (FIG. 7). The results showed that TGF-β1 and TGF-β R1 were significantly reduced in NHSC that were treated with BL100, BL250, and BL500 for 12 and 24 h as compared to that of normal control and curcumin-treated cells. Similar result of reduction was also observed in BL extract-treated THSC. Curcumin cannot reduce TGF-β1 in NHSC and THSC (except 24 h treatment), but significantly reduced TGF-β R1 in NHSC.

TGF-β/Smad signaling pathway is important for liver fibrosis. Next, we further investigated expression levels of Smad proteins in BL extract-treated NHSC and THSC (FIG. 8). The results showed that pSmad2 and Smad4 were significantly reduced in a dose dependent manner in NHSC and THSC that were treated with BL250 and BL500 for 12 and 24 h. pSmad 3 was significantly reduced in NHSC that were treated with BL250 (24 h treatment) and BL500 (12 and 24 h treatment). Smad7 was also significantly reduced in NHSC and THSC (except 12 h treatment) that were treated with BL500 for 12 and 24 h. Taken together, BL extract down regulate not only TGF-β1 and TGF-β R1, but also Smad proteins, such as pSmad2, Smad4 and Smad7.

### Example 7

### Col 1, α-SMA, MMP2 and TIMP2 were Significantly Reduced in BL Extract-Treated NHSC and THSC

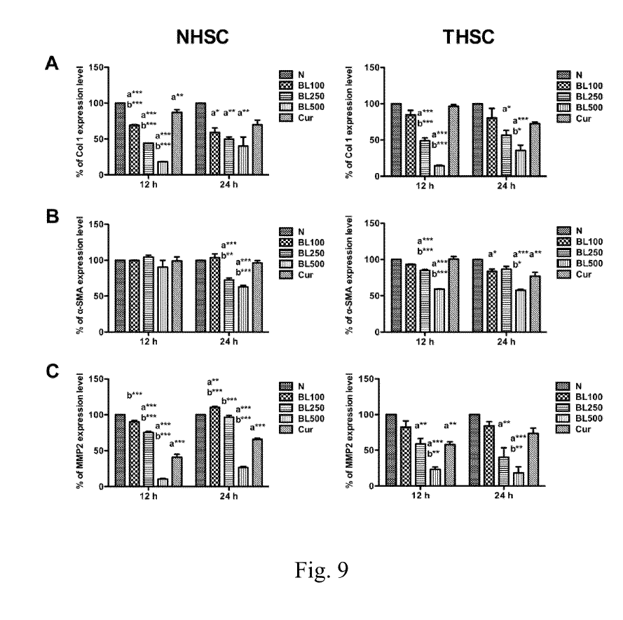

Liver fibrosis is characterized by excessive accumulation of ECM, including Col 1, α-SMA, and F-actin stress fibers that are secreted by activated HSC. According to our recently results in Example 5, F-actin stress fibers were reduced in BL extract-treated NHSC and THSC (FIG. 6). We further analyzed whether BL extract influence expression levels of Col 1 and α-SMA in NHSC and THSC (FIGS. 9A and B). NHSC and THSC that were treated with BL for 12 and 24 h showed a significant reduction in Col 1 and α-SMA (except 12 h treatment in NHSC) levels as compared to that of normal control. Curcumin failed to reduce expression levels of Col 1 and α-SMA in NHSC and THSC (except 24 h treatment).

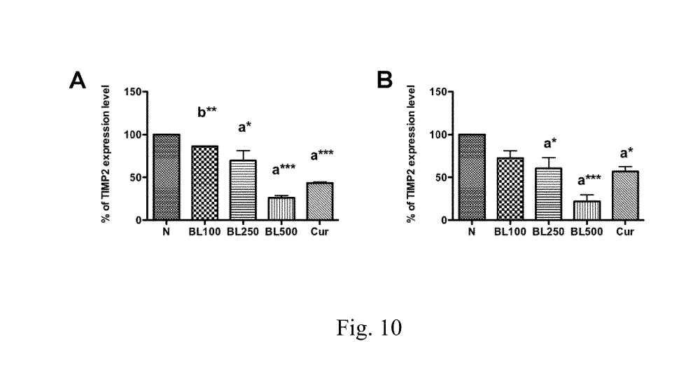

In liver fibrosis, TIMP2 and MMP-2 are highly expressed in activated HSC and may have a profibrogenic role. We further analyzed whether BL extract influence expression levels of MMP-2 (FIG. 9C) and TIMP2 (FIG. 10) in NHSC and THSC. The results showed that MMP2 level was significantly decreased in a dose-dependent manner in NHSC and THSC that were treated with BL for 12 and 24 h as compared to that of normal control. Curcumin can reduce expression level of MMP2 in NHSC and THSC (except 24 h treatment). The expression level of TIMP2 was also significantly reduced in NHSC and THSC that were treated with BL250, BL500, and curcumin compared to that of normal control. Taken together, BL extract not only influenced expression levels of Col 1 and α-SMA but also down regulate MMP2 and TIMP2 levels that are important for liver fibrosis.

### REFERENCE

1. Balunas M J, Kinghorn A D. 2005. Drug discovery from medicinal plants. Life Sci 78:431-441.
2. Chang K T, Tsai M J, Cheng Y T, Chen J J, Hsia R H, Lo Y S, Ma Y R, Weng C F. 2009. Comparative atomic force and scanning electron microscopy: an investigation of structural differentiation of hepatic stellate cells. Journal of structural biology 167:200-208.
3. Chen M, Jin H Z, Zhang W D, Yan S K, Shen Y H. 2009. Chemical constituents of plants from the genus Blumea. Chem Biodivers 6:809-817.
4. Chiang L C, Cheng H Y, Chen C C, Lin C C. 2004. In vitro anti-leukemic and antiviral activities of traditionally used medicinal plants in Taiwan. Am J Chin Med 32:695-704.
5. Farazi P A, DePinho R A. 2006. Hepatocellular carcinoma pathogenesis: from genes to environment. Nat Rev Cancer 6:674-687.
6. Forbes S J, Russo F P, Rey V, Burra P, Rugge M, Wright N A, Alison M R. 2004. A significant proportion of myofibroblasts are of bone marrow origin in human liver fibrosis. Gastroenterology 126:955-963.
7. Friedman S L, Roll F J, Boyles J, Bissell D M. 1985. Hepatic lipocytes: the principal collagen-producing cells of normal rat liver. Proc Natl Acad Sci USA 82:8681-8685.
8. Goldman P. 2001. Herbal medicines today and the roots of modern pharmacology. Ann Intern Med 135:594-600.
9. Kinnman N, Francoz C, Barbu V, Wendum D, Rey C, Hultcrantz R, Poupon R, Housset C. 2003. The myofibroblastic conversion of peribiliary fibrogenic cells distinct from hepatic stellate cells is stimulated by platelet-derived growth factor during liver fibrogenesis. Lab Invest 83:163-173.
10. Malarkey D E, Johnson K, Ryan L, Boorman G, Maronpot R R. 2005. New insights into functional aspects of liver morphology. Toxicol Pathol 33:27-34.
11. Ragasa C Y, Wong J, Rideout J A. 2007. Monoterpene glycoside and flavonoids from Blumea lacera. J Nat Med-Tokyo 61:474-475.
12. Reagan-Shaw S, Nihal M, Ahmad N. 2008. Dose translation from animal to human studies revisited. Faseb J 22:659-661.
13. Roberts R A, Ganey P E, Ju C, Kamendulis L M, Rusyn I, Klaunig J E. 2007. Role of the Kupffer cell in mediating hepatic toxicity and carcinogenesis. Toxicological sciences: an official journal of the Society of Toxicology 96:2-15.
14. Santi-Rocca J, Rigothier M C, Guillen N. 2009. Host-microbe interactions and defense mechanisms in the development of amoebic liver abscesses. Clinical microbiology reviews 22:65-75.
15. Uddin S J, Grice I D, Tiralongo E. 2011. Cytotoxic effects of bangladeshi medicinal plant extracts. Evid Based Complement Alternat Med 2011:578092.

1. A method for liver fibrosis prevention and/or treatment, comprising: administrating an effective amount of a Blumea lacera extract to an object.

2. The method of claim 1, wherein said effective amount is 1 to 5 g/60 kg body weight/day.

3. The method of claim 1, wherein said effective amount is 100 to 500 μg/ml under in vitro cell culture experiment; wherein said effective amount is based on the volume of culture medium used in each culture of said in vitro cell culture experiment.

4. The method of claim 1, wherein said liver fibrosis prevention and/or treatment comprising accumulating lipid in hepatic stellate cells, down-regulating the proliferation of hepatic stellate cells, inhibiting the mobility of hepatic stellate cells, preventing the activation of hepatic stellate cells, decreasing the synthesis of ECM proteins, or a combination thereof.

5. The method of claim 1, wherein the route of said administrating is via oral administration, intravenous injection, or a combination thereof.

6. The method of claim 1, wherein said Blumea lacera extract is an alcohol extract of Blumea lacera.

7. The method of claim 6, wherein said alcohol extract is an ethanol extract.

8. The method of claim 1, wherein said Blumea lacera extract is made by an extraction process; wherein said extraction process comprises the following steps:
obtaining a Blumea lacera plant;
soaking said Blumea lacera with an ethanol to obtain a mixture;
concentrating said mixture to form said extract.

9. The method of claim 8, wherein said Blumea lacera plant is a whole plant of said Blumea lacera.

10. The method of claim 8, wherein said soaking is achieved by putting said Blumea lacera into a bath of said ethanol.

11. The method of claim 8, wherein said ethanol is of a concentration of 90 to 95% (v/v).

12. The method of claim 8, wherein said soaking is performed for 120 to 168 hours.

13. The method of claim 8, wherein said soaking is performed at 26 to 28° C.

14. The method of claim 8, wherein said mixture is filtered before being concentrated.

15. The method of claim 8, wherein said concentrating is achieved by reduced pressure.

16. The method of claim 8, wherein said extraction process further comprises the step of dissolving said extract in DMSO, absolute ethanol, anhydrous ethanol, or a combination thereof after concentration.

17-20. (canceled)
